(19) United States
(12) Patent Application Publication
(10) Pub. No.: US 2026/0242743 A1
(43) Pub. Date: Aug. 20, 2026

(54) METHODS FOR CULTURING ALVEOLAR EPITHELIAL CELLS

(71) Applicant: STEMCELL TECHNOLOGIES CANADA INC., Vancouver (CA)

(72) Inventors: Natalie RONAGHAN, Vancouver (CA); Philipp KRAMER, Vancouver (CA)

(21) Appl. No.: 19/164,290

(22) PCT Filed: Mar. 28, 2024
(86) PCT No.: PCT/CA2024/050405

§ 371 Date: Sep. 11, 2025

Related U.S. Application Data
(60) Provisional application No. 63/455,678, filed on Mar. 30, 2023.

(51) Int. Cl. C12N 5/071

(52) U.S. Cl. CPC C12N 5/0688; C12N 2506/27; C12N 2533/50; C12N 2533/52; C12N 2533/54

(57) ABSTRACT

Disclosed are methods, media, supplements and kits for expanding, maintaining, or differentiating mammalian epithelial cells, such as alveolar epithelial cells—AT1 and AT2 cells, in monolayer. The methods of this disclosure can be used to expand, maintain or differentiate mammalian alveolar epithelial cells in a 2D environment when such cells are seeded in the presence of one or more extracellular matrix proteins.

### CROSS-REFERENCE TO RELATED APPLICATION

This application claims the benefit of U.S. Provisional Patent Application No. 63/455,678, filed Mar. 30, 2023, the entire contents of which is hereby incorporated by reference in its entirety.

### TECHNICAL FIELD

The present disclosure relates to cell culture applications, and more specifically to cell culture applications using mammalian alveolar epithelial cells, and still more specifically to cell culture applications related to a population(s) of alveolar epithelial Type 1 and Type 2 cells.

### BACKGROUND

The culture of pulmonary cells is the subject of much past, and increased current, research. In particular, the alveolar region of lungs is of intense interest due to its involvement in gas exchange, and its role in various infections and pathologies.

While the culture of alveolar organoids has recently emerged as a good model system to study certain infections and pathologies, the culture of cells of the alveolar region in monolayer may provide a model having certain advantages over organoid models. For example, monolayer culture conditions may provide better or easier access to the apical side, and also more closely mimic the in vivo situation through direct exposure to air in air-liquid interface culture systems. However, the ability to prepare such monolayer cultures suffer various challenges, such as providing sufficient cell attachment conditions, maintaining appropriate cell phenotypes, and obtaining sufficient numbers of cells.

Therefore, there is a need to develop monolayer culture systems that enable the exploration of research questions relating to alveolar infections and pathologies.

### SUMMARY

The present disclosure relates to methods, culture media, supplements, and kits for expanding, maintaining and/or differentiating alveolar epithelial cells.

In one aspect of this disclosure are provided methods for culturing epithelial cells in monolayer. The methods of this disclosure may comprise, a) seeding a population of epithelial cells onto a surface coated with one or more extracellular matrix proteins, b) exposing the population of seeded cells to a cell culture medium for a sufficient culture time, and c) yielding a culture of epithelial cells in monolayer. In one embodiment, the one or more extracellular matrix proteins comprise one or more of a collagen, a vitronectin, or a laminin.

In one embodiment, the surface is a bottom wall of a plate or a membrane of a cell culture insert, such as a Transwell culture insert.

In one embodiment, methods of this disclosure further comprise seeding the population of cells onto a surface coated with two or more extracellular matrix proteins. In one embodiment, the two or more extracellular matrix proteins are a laminin and a collagen.

In one embodiment, the laminin is laminin 332, laminin 111 or laminin 521 and the collagen is collagen type I, type II, type III or type IV.

In one embodiment, the concentration of the one or more extracellular matrix proteins ranges between 1 μg/mL to 1 mg/mL.

In one embodiment, methods of this disclosure further comprise culturing the population of cells submerged in the culture medium or in air-liquid interface conditions.

In one embodiment, the epithelial cells are alveolar epithelial cells. In one embodiment, the alveolar epithelial cells are alveolar epithelial type 2 (AT2) cells and/or alveolar epithelial type 1 (AT1) cells.

In one embodiment, the population of cells are donor- or PSC-derived. In one embodiment, the population of cells is of mammalian origin, such as a human or rodent origin. In one embodiment, the population of cells are cryopreserved or freshly isolated.

In one embodiment, the population of cells are a suspension of cells dissociated from an organoid. In one embodiment, the organoid is an alveolar organoid.

In one embodiment, the culture time is 3-14 days.

In one embodiment, the cell culture medium is an expansion medium.

In one embodiment, methods of this disclosure further comprise d) differentiating the culture of cells from alveolar epithelial type 2 cells to alveolar epithelial type 1 cells in a cell culture medium.

In one embodiment, the cell culture medium for differentiating the culture of cells from alveolar epithelial type 2 cells to alveolar epithelial type 1 cells is a differentiation medium.

In one embodiment, methods of this disclosure further comprise providing a pre-differentiated population of cells before seeding in step a) above. In one embodiment, the pre-differentiated population of cells comprise alveolar epithelial type 1 cells differentiated from alveolar epithelial type 2 cells.

In one embodiment, the pre-differentiated population of cells are differentiated in a dome culture system for a sufficient period of time. In one embodiment, the dome culture is for 5-14 days.

In another embodiment, the disclosure comprises a kit for culturing mammalian epithelial cells as an adhered culture. In one embodiment, the kit comprises:
a) at least one container comprising a basal medium;
b) at least one container comprising a first supplement, the first supplement comprising at least one cytokine and at least one growth factor;
c) at least one container comprising a first extracellular matrix protein;
d) optionally, at least one container comprising a second extracellular matrix protein, wherein the first extracellular matrix protein and the second extracellular matrix protein are comprised in the same or different at least one container;
e) optionally, at least one cell culture vessel;
f) optionally, at least one cell culture insert to be received into a receptacle of the at least one cell culture vessel; and
g) optionally, at least one container comprising epithelial cells, wherein the epithelial cells are a suspension of cells or comprised in an organoid.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows expansion and maintenance of AT2 cells cultured in a 2D (e.g. monolayer) environment. Representative microscopic images of AT2 cell expansion and maintenance when AT2 cells from two different human donors were expanded for 7 days in submerged culture conditions, testing the effects of different individual (Conditions 1, 2, and 3) and pairwise combinations (Conditions 4 and 5) of extracellular matrix proteins (A). Long-term (day 20) maintenance of AT2 cells culture from a human donor in the same submerged culture conditions as in panel A) (B). Representative microscopic images of AT2 cell expansion in from three different human donors on the extracellular matrix proteins of condition 5 in A) and B), cultured at either air-liquid interface (ALI) for 3 or 4 days after confluency or in submerged culture for 7 or 8 days (C). Scale bars in A), B) and C) represent 200 μm. Bar graph of flow cytometry data summarizing the percentage of viable singlets expressing AT2 (HT2-280 hi+) and AT1 (HT1-56 hi+) markers after AT2 cells from three different human donors were expanded in the presence of an individual (Condition 2) or a combination of two (Condition 5) extracellular matrix proteins, and either at air-liquid interface (ALI) for 3 or 4 days after confluency or in submerged culture (sub) for 7 or 8 days. Percentages of viable singlets from a 3D Matrigel dome control of AT2 cells are shown (D). Bars represent mean ±SEM of three donors.

FIG. 2 shows differentiation data of AT1 cells cultured in 2D. Representative microscopic images of AT1 cells differentiated in submerged culture from AT2 cells of the two human donors of FIG. 1A, either in contact with different individual (Conditions 1 and 2) or different pairwise combinations (Conditions 4 and 5) of extracellular matrix proteins (A). Conditions 1, 2, 4 and 5 are the same as in FIG. 1. Representative microscopic images of AT1 cells differentiated from AT2 cells of the three human donors of FIG. 1B following expansion on two extracellular matrix proteins (Condition 5), and in either air-liquid interface (ALI) or submerged culture conditions (B). Scale bars in A) and B) represent 200 μm. Bar graph of flow cytometry data summarizing the percentage of viable singlets of AT1 (HT1-56 hi+) cells differentiated from AT2 cells of three different human donors. AT2 cells were expanded and differentiated in the presence of an individual (Condition 2) or a combination of two extracellular matrix proteins (Condition 5), and either at air-liquid interface (ALI) for 3 or 4 days after confluency or in submerged culture for 7 or 8 days. Percentages from a 3D Matrigel dome control of AT1 cell differentiation are shown (C). Bars represent mean±SEM of three donors.

FIG. 3 shows the results of immunofluorescence staining for AT2 and AT1 marker expression by immunocytochemistry (ICC). AT2 cells from a single human donor were cultured in 2D in the presence of two extracellular matrix proteins (Condition 5) and either at air-liquid interface (ALI) or in submerged culture for up to 14 days. Cells were collected on day 7 and day 14 for ICC. Four-channel immunofluorescence images of the same focal area for each condition tested show AT2-related HT2-280expression (white arrows), AT1-related RAGE and GPRC5a expression (white arrowheads and open arrows, respectively), and DAPI for nuclei (circled). Scale bars represent 25 μm.

### DETAILED DESCRIPTION

This disclosure relates to media compositions and/or media supplements and/or kits (comprising media and supplements), and to methods for expanding and/or maintaining epithelial cells (e.g., alveolar epithelial cells). In one embodiment, this disclosure relates to media compositions and/or media supplements and/or kits (comprising media and supplements), and to methods for differentiating epithelial cells (e.g., alveolar epithelial cells). In one embodiment, this disclosure relates to (in vitro) expansion and/or differentiation of epithelial cells such as alveolar epithelial cells in 2D using media, supplements, and kits of this disclosure, or by practicing the disclosed methods.

Where used in this disclosure, the term “epithelial cells” refers to cells that cover the body surface (e.g. skin), inner walls of cavities in organs, and blood vessels. Exemplary epithelial cells include those found in the epithelium of lungs (bronchi, alveoli), intestines (stomach, duodenum, small intestine, large intestine), oral and nasal cavities, tongue, lymphatic vessels, serosal membranes (pericardium, pleura, peritoneum, etc.), renal collecting tubules, thyroid glands, blood vessels, liver, salivary glands, skin epidermis, esophagus, vagina, sweat glands, germinal epithelium, testicular epithelium, follicles, exocrine glands, ureters, and bladder. Epithelial cells of this disclosure may be primary-derived, and may be sourced from any ethically obtained organ, including but not limited to, liver, lung, pancreas or intestine. In one embodiment, epithelial cells are obtained from an epithelial fragment (e.g. a tissue/organ fragment comprising one or more epithelial stem/progenitor cells). In one embodiment, epithelial cells are obtained or isolated from an (epithelial) organoid (e.g. a three-dimensional in vitro model of an organ).

Where used in this disclosure, the term “alveolar epithelial cells”—used interchangeably with other terms such as “pulmonary alveolar epithelial cells” and “pneumocytes”—refers to epithelial cells that in vivo are positioned on the basement membrane of alveoli, such as type 1 and type 2 alveolar epithelial cells. Type 1 alveolar cells (over 95% of the population) are large squamous cells involved in gas exchange between alveoli and blood. Primary alveolar epithelial type I cells (e.g. “AT1” or “ATI”) may express AT1 cell markers such as AQP5, AGER, HOPX, PDPN, CAV1, SCNN1A, AKAP5, and CLIC5. AT1 cells can be detected by HT1-56, a biomarker specific to the apical plasma membrane of human lung alveolar type 1 cells. Type 2 alveolar cells (e.g. “AT2” or “ATII”) (between 2-5% of the population) produce pulmonary surfactant protein C (SPC) and have characteristic morphological features, such as lamellae and multivesicular bodies. Primary alveolar epithelial type 2 cells (e.g. “ATII” or “AT2”) are characterized by high expression of, e.g., SFTPC, SPTPB, SFTPA1 ABCA3, and ICAM-1. AT2 cells can be detected by HT2-280, a biomarker specific to the apical plasma membrane of human lung alveolar type 2 cells. While type 1 alveolar epithelial cells are generally unable to replicate, type 2 alveolar epithelial cells proliferate and may differentiate to other cell types, including type I alveolar epithelial cells.

Where used in this disclosure, the term “extracellular matrix”, “extracellular matrix protein” or “ECM” refers to extracellular molecules that provide structural and biochemical support to surrounding cells. An extracellular matrix may comprise one or more extracellular matrix proteins that promote cell adhesion, cell-to-cell communication and differentiation within a given tissue or agglomerate of cells. Both natural and synthetic extracellular matrices or proteins are contemplated within the present disclosure. Components of an extracellular matrix, and thus an extracellular matrix within the scope of this application, may include one or more of the following proteins: a fibronectin, a laminin, a vitronectin, a tenascin, an entactin, a thrombospondin, an elastin, a gelatin, a collagen, a fibrillin, a merosin, an anchorin, a chondronectin, a link protein, a bone sialoprotein, an osteocalcin, an osteopontin, an epinectin, a hyaluronectin, an undulin, an epiligrin, a kalinin, a synthetic polymer-based hydrogel (such as a polyethylene glycol, a polyvinyl, or any derivative or analogue thereof), a plant-based hydrogel (such as a cellulose, a hemicellulose, a lignin, a starch and a pectin, or any derivative or analogue thereof), a proteoglycan (e.g., heparan sulfate, chondroitin sulfate, keratin sulfate), hyaluronic acid. Many collagens are known, such as fibrillar (Type I, II, III, V, XI), FACIT collagen (Fibril Associated Collagens with Interrupted Triple helices) (Type IX, XII, XIV, XIX, XXI collagen and collagen type XXII alpha 1), short chain (collagen Type VIII and X), basement membrane (collagen Type IV), and Type VI, VII, XII collagen). In one embodiment, an extracellular matrix used in or with a culture medium described herein is a gelatinous protein mixture secreted by cells, such as fibroblasts, chondrocytes, or Engelbreth-Holm-Swarm (EHS) mouse sarcoma cells. In some embodiments, the extracellular matrix protein is Matrigel™. ECM proteins of this disclosure may be comprised in a medium of this disclosure or comprised in a supplement to be added thereto, or they may be coated on a cell culture surface, such as that of a vessel or Transwell® insert.

Where used in this disclosure, the term “cell culture medium” refers to a liquid, semi-liquid or gelatinous substance containing nutrients in which cells or tissues can be cultivated (e.g., expanded, maintained, or differentiated). Cell culture media typically supply components required by cells to meet nutritional needs as they proliferate, differentiate, or are maintained in vitro. The term “expansion medium” refers to a cell culture medium that can support the proliferation/expansion of cells in vitro, such as stem cells. In one embodiment, the cell culture medium is a lung stem cell (e.g. AT2) expansion medium, such as may be sold under the PneumaCult™ (STEMCELL Technologies) or the STEMdiff™ (STEMCELL Technologies) brands. In one embodiment, an expansion medium supports in vitro proliferation and expansion of AT1 cells. The term “maintenance medium” refers to a cell culture medium that can maintain a particular cell state in culture. In one embodiment, a maintenance medium maintains AT2 cell identity, and optionally represses induction of AT1 cells. In one embodiment, a maintenance medium maintains AT1 cell identity, and optionally represses induction of AT2 cells. The term “differentiation medium” refers to a cell culture medium that can promote a cell to differentiate from a particular cell state into a different cell state. In one embodiment, a differentiation medium of the present disclosure differentiates AT2 cells to AT1 cells.

Cell culture media of this disclosure (e.g. expansion, maintenance, and differentiation) may comprise a basal medium, and such basal medium may be the same or different depending on whether it is used for expansion, maintenance or differentiation. In one embodiment, the basal medium requires supplementation with one or more additives to formulate a complete medium. In one embodiment, media of this disclosure are used to culture cells at the air-liquid interface and/or in submerged culture. In one embodiment, media of this disclosure may be used to culture epithelial cells. In one embodiment, media of this disclosure may be used to culture non-epithelial cells. In one embodiment, media of this disclosure may be used to grow or maintain immune cells.

Where used in his disclosure, the term “culture condition” refers to an environment in which cells, such as epithelial cells, may be expanded, maintained and/or differentiated. Thus, a culture condition comprises those features that establish the environment in which epithelial cells may be expanded, maintained, and/or differentiated, as the case may be. In one embodiment, a culture condition comprises at least a cell culture medium, one or more extracellular matrix proteins, and a cell culture vessel. In certain embodiments, a culture condition may also relate to the way cells are cultured (e.g. in suspension, as a monolayer, or in a cell culture insert either in “submerged” and/or at “air-liquid interface (ALI)” conditions. A submerged culture may comprise immersing or enveloping epithelial cells, such as alveolar cells, in a cell culture medium and/or an extracellular matrix. An air-liquid interface culture may comprise seeding epithelial cells, such as alveolar epithelial cells, onto a porous substrate, such that a top surface/plane of the cells is in contact with the air and a bottom surface/plane of the cells is in contact with a culture medium in contact with one side of the substrate. In one embodiment, cells may be sequentially cultured submerged then at ALI, or vice versa.

### Media and Supplements

In one aspect of this disclosure are provided media for expanding, maintaining, or differentiating (a population of) epithelial cells, such as alveolar epithelial cells adhered to a substrate (e.g. in monolayer). In one aspect of this disclosure are provided supplements to be added to media for expanding, maintaining and/or differentiating a population of epithelial cells, such as alveolar epithelial cells adhered to a substrate (e.g. in monolayer). In other aspects, media (and associated) supplements, one or more extracellular matrix proteins, and optionally cultureware (e.g. dishes/plates/cell culture inserts) of this disclosure may be comprised in a system or a kit.

Epithelial cells cultured in contact with media of this disclosure are not particularly limited and may correspond to any epithelial cell type that can be expanded, maintained and/or differentiated. In a specific embodiment, epithelial cells are pulmonary, and more specifically may be obtained from pulmonary alveolar epithelium.

Where the epithelial cells are alveolar epithelial cells, the alveolar epithelial cells may be AT1 and/or AT2 cells. In one embodiment, the AT1 and/or AT2 cells are human. In one embodiment, the AT1 and/or AT2 cells are donor-derived. In one embodiment, the AT1 and/or AT2 cells are freshly isolated cells. In one embodiment, the AT1 and/or the AT2 cells are cryopreserved.

In one embodiment, the epithelial cells (e.g. alveolar epithelial cells) are pluripotent stem cell (PSC)-derived. In one embodiment, the PSCs are embryonic stem cells (ESC) or induced pluripotent stem cells (iPSC). If the epithelial cells (e.g. alveolar epithelial cells) are PSC-derived, they may be human, non-human primate, livestock, canine, feline, rodent, or otherwise.

Regardless of whether culture media of this disclosure are expansion, maintenance, or differentiation media, they may comprise a basal medium. Basal media comprised in expansion, maintenance, or differentiation media may be the same, or may be different.

As contemplated herein, a basal medium is not particularly limited provided that it is capable (when appropriately supplemented) of supporting the expansion and/or maintenance of epithelial cells (e.g., AT2 and/or AT1 cells), and/or supporting the differentiation of epithelial cells (e.g., AT2 cells). In a specific embodiment, a basal medium supports the expansion and/or maintenance (when appropriately supplemented) of alveolar epithelial cells (e.g., AT2 and/or AT1 cells), and/or supporting the differentiation of alveolar epithelial cells (e.g., AT2 cells).

Basal media are well known in the art, and many of which are commercially available. Examples of basal media may include RPMI, DMEM/F-12, IMDM, Advanced DMEM, Advanced DMEM/F-12, and PneumaCult™-branded media. Basal media typically include one or more of a carbohydrate, amino acids, trace elements, lipid(s), buffer(s), salt(s), vitamin(s), nucleoside(s), and the like for supporting or maintaining the growth of cells. In some embodiments, basal media do not include one or more of the foregoing components, and if essential may be supplemented.

A basal medium used to formulate an epithelial (e.g. alveolar) expansion, maintenance and/or differentiation medium of this disclosure may be supplemented with appropriate, additional components to make a complete medium. In one embodiment, a basal medium is combined with one or more supplements to form a complete medium. In one embodiment, the basal medium is provided together (whether in a kit or otherwise) with one or more supplements to be added thereto prior to use. In one embodiment, the one or more supplements can be concentrated (for e.g., 10×, 100×) in a suitable liquid and brought to a working concentration through combination with an appropriate diluent, such as a buffer, medium, water, or any inorganic solvent known to a person skilled in the art.

In addition to a basal medium, complete expansion, maintenance, and/or differentiation media of this disclosure may further comprise one or more of: growth factor(s), protein(s), cytokine(s), lipid(s), hormone(s), salt(s), vitamin(s), one or more source of an albumin, and small molecule(s).

In one embodiment, culture media and/or supplements of this disclosure may comprise at least one cytokine and/or at least one growth factor. In one embodiment, culture media and/or supplements may comprise one or more cytokines. Examples of cytokines include, but are not limited to interleukin-Ia (IL-Ia), interleukin-1β (IL-1β), interleukin-2 (IL-2), interleukin-3 (IL-3), interleukin-4 (IL-4), interleukin-5 (IL-5), interleukin-6 (IL-6), interleukin-7 (IL-7), interleukin-8 (IL-8), interleukin-9 (IL-9), interleukin-10 (IL-10), interleukin-11 (IL-11), interleukin-12 (IL-12), interleukin-13 (IL-13), interleukin-14(IL-14), interleukin-15 (IL-15), interleukin-16 (IL-16), interleukin-17 (IL-17), interleukin-17 (IL-18), INF-α, INF-β, INF-γ, and tumor necrosis factor-a (TNF-a). In one embodiment, culture media and/or supplements may comprise one or more growth factors, such as one or more of a mitogen (e.g. EGF, an NRG, AREG, etc), an FGF, an IGF, etc. In some embodiments, culture media and/or supplements may comprise a ROCK (Rho kinase) inhibitor. Examples of ROCK inhibitors include, but are not limited to, Y27632, Ripasudil (K-I 15), Netarsudil (AR-13503), RKI-18, and RKI-11. In one embodiment, the concentration of one or more cytokine or growth factor may be between about 0.5 ng/ml and 500 ng/ml, between about 1 ng/ml and 250 ng/mL, between about 5 ng/ml and 100 ng/mL, or between about 10 ng/ml and 50 ng/ml.

Where culture media and/or supplements of this disclosure are used to seed a population of cells, such as alveolar epithelial cells, such media/supplements may comprise one or more cytokines and/or one or more growth factors. In one embodiment, the one or more cytokines may include interleukin-1β (IL-1β). Seeding media may further comprise a ROCK (Rho kinase) inhibitor (e. g Y27632).

Where culture media and/or supplements of this disclosure are used to expand a population of cells, such as alveolar epithelial cells, such media/supplements may comprise one or more cytokines and/or one or more growth factors. In one embodiment, the one or more cytokines may include interleukin-1β (IL-1β) and/or an epidermal growth factor (EGF) and/or a fibroblast growth factor (FGF, e.g. FGF10). Expansion media may further comprise an activator of wnt signalling, such as CHIR, and/or an inhibitor of TGF signaling. Expansion media may further comprise a ROCK (Rho kinase) inhibitor (e.g Y27632).

Where culture media and/or supplements of this disclosure are used to differentiate a population of cells, such as alveolar epithelial cells, such media/supplements may comprise one or more cytokines and/or one or more growth factors. In one embodiment, the one or more cytokines may include an epidermal growth factor (EGF) and/or a fibroblast growth factor (FGF, e.g. FGF10).

In one embodiment, an expansion, maintenance, or differentiation medium is serum-free. In one embodiment, an expansion, maintenance, or differentiation medium is serum-containing.

Media (e.g. expansion, maintenance, and/or differentiation) of this disclosure may form a culture environment or culture condition with one or more extracellular matrix proteins. In one embodiment, one or more extracellular matrix proteins may be comprised in a medium. In one embodiment, one or more extracellular matrix proteins may be coated on a cell culture surface that comes into contact with a medium. In one embodiment, two or more extracellular matrix proteins may be comprised in a medium. In one embodiment, two or more extracellular matrix proteins may be coated on a cell culture surface that comes into contact with a medium.

Extracellular matrix protein(s) of this disclosure are not particularly limited, and in specific embodiments may be one or more of a collagen, an ECM1, a laminin, an osteopontin, a vitronectin, a fibronectin. In one embodiment, extracellular matrix proteins are comprised in a natural or synthetic extracellular matrix, such as hydrogel or Matrigel™.

Extracellular matrix protein(s) may be recombinant or of natural origin (e.g. purified from a biological sample). In one embodiment, some but not all of the extracellular matrix proteins are recombinant. In one embodiment, some or all of the recombinant extracellular matrix proteins are human and/or mouse. In one embodiment, some or all of such extracellular matrix proteins are genetically engineered. In one embodiment, some or all of such extracellular matrix proteins are a whole protein or a fragment thereof, such as a peptide fragment.

In embodiments comprising a laminin, the laminin may be one or more of laminin-511, laminin-552 (also known as laminin 11), laminin-332, or a fragment thereof. In embodiments comprising a collagen, the collagen may be one or more of collagen type 1, collagen type II, collagen type III, or collagen type IV. In one embodiment, the extracellular matrix protein is collagen I or collagen III. In one embodiment, the one or more extracellular matrix proteins comprise a laminin and a collagen. In one embodiment, the one or more extracellular matrix proteins comprise laminin 332 and collagen III.

In one embodiment, the one or more extracellular matrix proteins are coated or immobilized on a surface of a culture receptacle or vessel in (or on) which epithelial cells (e.g. alveolar epithelial cells) are contained or cultured, such as a culture plate, a culture plate insert (e.g. a Transwell™ insert), a membrane (e.g. a porous membrane), a dish, a well, or a bottle. Thus, in such an embodiment, a cell cultured in or on the receptacle is cultured in the presence of a medium of this disclosure and in the presence of one or more extracellular matrix proteins that are coated or immobilized on a surface thereof. In one embodiment, the surface of a culture receptacle or vessel is cell culture treated. In one embodiment, the surface of a culture receptacle or vessel is not cell culture treated.

In one embodiment, a culture receptacle or vessel surface may be coated by layering two or more (individual) extracellular matrix proteins. In one embodiment, a culture receptacle or vessel surface is first coated with a first type of extracellular matrix protein, and then layered with a second type of extracellular matrix protein.

In one embodiment, extracellular matrix protein(s) coated on a culture receptacle or vessel may be diluted in a suitable buffer before use.

Regardless of whether the one or more extracellular matrix proteins (whether single or combination of individual proteins) is comprised in a medium or coated/immobilized on a surface, its concentration may range between about 0.1 μg/mL to 1 mg/mL. In one embodiment, the concentration of the one or more extracellular matrix proteins (whether single or combination of individual proteins) ranges between about 1 μg/mL to 100 μg/mL, about 3 μg/mL to 50 μg/mL, about 5 μg/mL to 30 μg/mL or about 10 μg/mL to 20 μg/mL.

In another aspect, the present disclosure provides for a kit or kits for expanding and/or maintaining, or kits for differentiating and/or maintaining epithelial cells (e.g. alveolar epithelial cells), as an adhered culture, such as in a monolayer. Thus, a kit or kits may comprise a basal medium and one or more growth factor and/or small molecule supplements for formulating complete (e.g. expansion, differentiation, maintenance) medium. In one embodiment, a kit or kits of this disclosure may further comprise one or more extracellular matrix protein(s), either to be added to media formulated from kit components or coated/immobilized on a culture surface. In one embodiment, a kit may further comprise cultureware, such as one or more plate/dish, and optionally one or more cell culture inserts (e.g. Transwell™ insert or the like).

### Methods

In one aspect of this disclosure are provided methods of culturing mammalian epithelial cells (e.g. alveolar epithelial cells). In one embodiment, methods of this disclosure are in vitro methods. In one embodiment, methods of this disclosure comprise culturing epithelial cells (e.g. alveolar epithelial cells) adhered to a substrate, such as in a monolayer. In one embodiment, the methods involve expanding or differentiating epithelial cells (e.g. alveolar epithelial cells) adhered to a substrate, such as in a monolayer. In one embodiment, expanded or differentiated epithelial cells (e.g. alveolar epithelial cells) may be maintained adhered to a substrate, such as in a monolayer.

Methods of this disclosure may comprise seeding a population of cells onto a surface, and exposing the population of cells to a cell culture medium. A cell culture medium of this disclosure may be as described above.

The population of cells is not particularly limited, and may correspond to any epithelial cell type that can be expanded, maintained and/or differentiated. In a specific embodiment, epithelial cells are pulmonary, and more specifically may be obtained from pulmonary alveolar epithelium.

Where the epithelial cells are alveolar epithelial cells, the alveolar epithelial cells may be AT1 and/or AT2 cells. In one embodiment, the AT1 and/or AT2 cells are human. In one embodiment, the AT1 and/or AT2 cells are donor-derived. In one embodiment, the AT1 and/or AT2 cells are freshly isolated cells. In one embodiment, the AT1 and/or the AT2 cells are cryopreserved.

In one embodiment, the epithelial cells (e.g. alveolar epithelial cells) are pluripotent stem cell (PSC)-derived. In one embodiment, the PSCs are embryonic stem cells (ESC) or induced pluripotent stem cells (iPSC). If the epithelial cells (e.g. alveolar epithelial cells) are PSC-derived, they may be human, non-human primate, livestock, canine, feline, rodent, or otherwise.

In one embodiment, the population of cells are alveolar epithelial cells, such as AT2 cells or AT1 cells, or a mixture of the two. In one embodiment, the population of cells are a suspension of cells dissociated from an organoid, such as an alveolar organoid. In one embodiment, an alveolar organoid comprises numerous cell types of the alveolar region, including at least some AT2 cells. In one embodiment, an alveolar organoid comprises numerous cell types of the alveolar region, including at least some AT2 cells and at least some AT1 cells.

Methods of this disclosure may relate to expanding a population of AT2 cells after seeding onto a surface. In one embodiment, an expanded population of AT2 cells may be seeded onto and maintained on the surface.

In addition or in the alternative, methods of this disclosure may relate to differentiating a population of AT1 cells. In one embodiment, a pre-differentiated population of cells (e. g AT1 cells) is seeded onto a surface, and the seeded cells are maintained on the surface. In one embodiment, the population of cells seeded onto the surface are AT2 cells, and they are differentiated to AT1 cells while cultured in monolayer.

Methods of this disclosure may comprise seeding a population of cells, as described above, onto a surface comprising or coated with one or more extracellular matrix proteins. In one embodiment, methods of this disclosure may comprise seeding a population of cells, as described above, onto a surface comprising or coated with two or more extracellular matrix proteins.

A surface may be any surface that supports a population of cells having been seeded thereupon and adhered (directly or indirectly) thereto, such as in a monolayer. By way of non-limiting example, the surface may be a bottom-wall of a cell culture vessel, such as a dish, plate, flask, vessel, or an insert that is placed therein. Further, the surface may be a membrane, filter, or any other type of porous surface that supports a population of cells seeded thereon, such as a cell culture insert (e.g. Transwell™ insert, or the like) that is supported in a well of a cell culture dish.

In one embodiment, the one or more extracellular matrix proteins are coated or immobilized on the surface prior to receiving the population of cells. In one embodiment, the one or more extracellular matrix proteins may be comprised in a cell culture medium that is applied to a vessel (against the surface) together with the population of cells. In such an embodiment, the one or more extracellular matrix proteins may become coated on the surface as the population of cells are settling thereupon.

In one embodiment, the population of cells are seeded onto a surface coated with two or more extracellular matrix proteins. In such embodiments, the surface may be coated by layering two or more (individual) extracellular matrix proteins (sequentially). In one embodiment, the surface of a culture receptacle or vessel may first be coated with a first type of extracellular matrix protein and then layered with a second type of extracellular matrix protein.

As described above, extracellular matrix protein(s) of this disclosure are not particularly limited, and in specific embodiments may be one or more of a collagen, an ECM1, a laminin, an osteopontin, a vitronectin, a fibronectin. In one embodiment, extracellular matrix proteins are comprised in a natural or synthetic extracellular matrix, such as hydrogel or Matrigel™.

In embodiments comprising a laminin, the laminin may be one or more of laminin-511, laminin-552 (also known as laminin 11), laminin-332, or a fragment thereof. In embodiments comprising a collagen, the collagen may be one or more of collagen type 1, collagen type II, collagen type III, or collagen type IV. In one embodiment, the extracellular matrix protein is collagen I or collagen III. In one embodiment, the one or more extracellular matrix proteins comprise a laminin and a collagen. In one embodiment, the one or more extracellular matrix proteins comprise laminin 332 and collagen III.

In one embodiment, the one or more extracellular matrix proteins are coated or immobilized on a surface of a culture receptacle or vessel in (or on) which epithelial cells (e.g. alveolar epithelial cells) are contained or cultured, such as a culture plate, a culture plate insert (e.g. a Transwell™ insert), a membrane (e.g. a porous membrane), a dish, a well, or a bottle. Thus, in such an embodiment, a cell cultured in or on the receptacle is cultured in the presence of a medium of this disclosure and in the presence of one or more extracellular matrix proteins that are coated or immobilized on a surface thereof. In one embodiment, the surface of a culture receptacle or vessel is cell culture treated. In one embodiment, the surface of a culture receptacle or vessel is not cell culture treated.

In one embodiment, a culture receptacle or vessel surface may be coated by layering two or more (individual) extracellular matrix proteins. In one embodiment, a culture receptacle or vessel surface is first coated with a first type of extracellular matrix protein, and then layered with a second type of extracellular matrix protein.

Regardless of whether the one or more extracellular matrix proteins (whether single or combination of ECMs) is comprised in medium or coated/immobilized on a surface, its concentration may range between about 0.1 μg/mL to 1 mg/mL. In one embodiment, the concentration of the one or more extracellular matrix proteins (whether single or combination of ECMs) ranges between about 1 μg/mL to 100 μg/mL, about 3 μg/mL to 50 μg/mL, about 5 μg/mL to 30 μg/mL or about 10 μg/mL to 20 μg/mL.

Methods of this disclosure may comprise seeding the population of cells on a two-dimensional support material or substrate, or embedded in a three-dimensional support material. Regardless, such support material(s) may cooperate with a culture medium to create a culture condition or environment. Indeed, culture media used in the methods of this disclosure may be as described above.

In embodiments, where a population of cells (e.g. AT2 and/or AT1 cells) is to be expanded after having been seeded on or within a coating applied to a surface, a culture medium will be appropriately formulated (e.g. an expansion medium) to expand the cells. In embodiments, where a population of cells (e.g. AT2) is to be differentiated (to AT1 cells) after having been seeded on or within a coating applied to a surface, a culture medium will be appropriately formulated (e.g. a differentiation medium) to differentiate the cells. In embodiments, where an expanded or differentiated population of cells is to be maintained on or in a coating applied to a surface, a culture medium will be appropriately formulated (e.g. a maintenance medium) to maintain the cells.

In one embodiment, methods of this disclosure are performed under animal component-free conditions (e.g. in an animal component-free culture environment). In the same or different embodiment, methods of this disclosure are performed under serum-free conditions (e.g. in a serum-free culture environment).

In one embodiment, an aspect of the culture condition or culture environment relates to culturing the seeded population of cells (while exposed to a culture medium) either at air-liquid interface (ALI) or in a submerged culture condition. In one embodiment of the methods disclosed herein, the seeded population of cells are cultured sequentially in either submerged culture and then moved to ALI, or vice versa.

In any of the foregoing culture conditions, a population of cells may also be seeded onto a surface coated with one or more extracellular matrix proteins, as described above. Nevertheless, exposure of the seeded population of cells to the culture medium (and the one or more extracellular matrix proteins) will be for a sufficient culture time to yield the desired cells. As described above, the desired (e.g. yielded) population of cells may be an expanded or differentiated population of epithelial cells (e.g. alveolar epithelial cells), and/or a maintained population of expanded or differentiated epithelial cells (e.g. alveolar epithelial cells). Also, as described above, in some embodiments the methods comprise yielding a culture of epithelial cells (e.g. alveolar epithelial cells) adhered (directly or indirectly) to a substrate, such as in a monolayer. In one embodiment, the yielded epithelial cells (e.g. alveolar epithelial cells) are alveolar epithelial type 2 cells. In one embodiment, the yielded epithelial cells (e.g. alveolar epithelial cells) are alveolar epithelial type 1 cells.

In one aspect, methods of this disclosure comprise a culture time that is sufficient to expand epithelial cells (e.g. alveolar epithelial cells) to a desired number, density or confluency. In one aspect, methods of this disclosure comprise a culture time that is sufficient to maintain epithelial cells (e.g. alveolar epithelial cells) at a desired number, density or confluency. In one aspect, methods of this disclosure comprise a culture time that is sufficient to differentiate epithelial cells (e.g. alveolar epithelial cells) to a desired number, density or confluency.

The culture time may be at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, or longer.

In embodiments where the methods yield a culture of epithelial cells (e.g. alveolar epithelial cells) adhered to a substrate and at air-liquid interface, the seeded population of cells may be exposed to the culture medium (and culture environment) for at least at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, or more.

In embodiments where the methods yield a culture of epithelial cells (e.g. alveolar epithelial cells) adhered to a substrate and in a submerged condition, the seeded population of cells may be exposed to the culture medium (and culture environment) for at least at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, or more.

In one embodiment, the culture time may be split into two stages (a first and a second stage). For example, the culture time of the first stage may be between 1-14 days in a first culture environment/condition (e.g. submerged culture condition). In one embodiment, the culture time of the first stage may be between about 1-5 days, or between about 3-14 days, in a first culture condition (e.g. submerged culture condition). Following a first stage, the culture time of the second stage may be between about 1-14 days in a second culture environment/condition (e.g. ALI condition). In one embodiment, the culture time of the second stage may be between about 1-5 days, or between about 3-14 days, in a second culture condition (e.g. ALI condition). In an alternative embodiment, the foregoing culture time may be as stated, but the first culture environment/condition is at ALI and the second culture environment/condition is submerged.

Prior to seeding the population of cells onto a coated surface (in a desired culture environment), the population of cells may in some embodiments be pre-differentiated. In one embodiment, the population of cells may be pre-differentiated to progenitor cells fated for a lineage of interest (e.g. the pulmonary lineage) from a population of stem cells, such as pluripotent stem cells. In one embodiment, the population of cells may be pre-differentiated primary cells, such as AT1 cells differentiated from AT2 cells.

In one embodiment, pre-differentiated AT1 cells may be prepared by embedding AT2 cells within an extracellular matrix (e.g. a dome) anchored to a bottom-wall or membrane of a culture vessel/insert, and exposed to differentiation (culture media) conditions. In one embodiment, in-dome differentiation comprises embedding a population comprising AT2 cells within a Matrigel® dome. In one embodiment, in-dome differentiation may be at least 1 day, at least 2 days, at least 3 days, at least 4 days, at least 5 days, at least 6 days, at least 7 days, at least 8 days, at least 9 days, at least 10 days, or longer.

Overall, the practice of the methods (and use of the media) of this disclosure may yield an expanded population of alveolar epithelial cells (e.g. AT2 and AT1 cells). More particularly, the practice of the methods (and use of the media) of this disclosure may yield up to a 100-fold, 200-fold, 500-fold, 1000-fold, or up to 5000-fold, or up to a 10,000-fold, or up to a 15,000-fold, or up to a 20,000-fold, or up to a 25,000-fold or higher increase in alveolar epithelial cells (e.g. AT2 and AT1 cells).

In one embodiment, the methods of this disclosure support the expansion of mammalian epithelial cells (e.g. alveolar epithelial cells). In one embodiment, the methods may support 5 or more, 10 or more, 15 or more, 20 or more, 25 or more, 30 or more, 35 or more, 40 or more, 45 or more, 50 or more, 55 or more, or 60 or more population doublings.

Following culture using media or practicing methods of this disclosure, a culture of epithelial cells (e.g. alveolar epithelial cells) in monolayer may be produced. In one embodiment, the culture of epithelial cells (e.g. alveolar epithelial cells) comprises a majority of alveolar epithelial type 2 (AT2) cells, such as >30%, >40%, >50%, >60%, >70%, >80%, or >90%. In one embodiment, the culture of epithelial cells (e.g. alveolar epithelial cells) in a culture condition/environment of this disclosure comprises more alveolar epithelial type 2 (AT2) cells than if the population of cells had not been cultured in a culture environment/condition as described herein. In one embodiment, the culture of alveolar epithelial cells comprises a majority of alveolar epithelial type 1 (AT1) cells, such as >30%, >40%, >50%, >60%, >70%, >80%, or >90%. In one embodiment, the culture of alveolar epithelial cells in a culture condition/environment of this disclosure comprises more alveolar epithelial type 1 (AT1) cells than if the population of cells had not been cultured in a culture environment/condition as described herein.

In one embodiment, the methods of this disclosure may comprise identifying the differentiated progeny of alveolar epithelial AT2 cells by detecting expression of at least one AT1 biomarker. In one embodiment, the methods may comprise differentiating AT2 cells wherein at least 60%, at least 70%, at least 80%, at least 90%, or at least 95% of the cells exhibit AT1 phenotype.

Epithelial cells (e.g. AT2 and AT1 cells) that have been expanded, maintained and/or differentiated in accordance with methods of this disclosure may be used in various assays or downstream applications. In one embodiment, the cells may be used in assays or methods to study viral or other infection, or injury. Thus, such methods may comprise the steps described herein, and additionally a step of exposing the culture of cells to a virus, pathogen or insult, and optionally to a medicament or a library of putative medicaments to control or resolve the infection, pathology, or injury.

In another embodiment, the cells may be used in assays or methods to study toxicity or in drug screening assays. Thus, such methods may comprise the steps described herein, and additionally a step of exposing the culture of cells to one or more compounds or drugs (test compound) and one or more control compound, and optionally to measure survival or death of the culture exposed to the test compound relative to those exposed to the control culture.

In another embodiment, the cells may be used for co-culture with endothelial, immune, or other cells. Thus, such methods may comprise the steps described herein, and additionally a step of combining one or more different cell types, whether into the same or different compartments of a culture well or environment (e.g. in apical and/or basolateral compartments of a Transwell™ insert, or in organ-on-chip systems). Thus, this disclosure contemplates methods of generating such model systems, and also to methods of modelling diseases (e.g. fibrosis, COVID, Crohn's or IBF, etc) and testing medicaments or putative medicaments, compounds or library of compounds.

Thus, 2D systems comprising relevant cell types that are accessible for assaying or further manipulation, may enable research and discovery of new treatment or therapeutic modalities/approaches.

The following non-limiting examples are illustrative of the present disclosure.

### EXAMPLES

### Example 1: Primary Human Epithelial Alveolar Cells

All cells used in this disclosure were obtained from human donors in accordance with applicable IRB and ethics requirements.

Human primary isolated or cryopreserved AT2 cells obtained from academic collaborators were processed as follows. Cryopreserved cells were thawed and transferred into a 15 ml conical tube containing 5-10 times complete PneumaCult™ Alveolar Organoid (AvO) Expansion Medium (EM) (STEMCELL Technologies). The tube was centrifuged at 400×g for 5 minutes at 2-8° C., and supernatant removed. The resulting pellet was resuspended in 5 mL cold AvO EM, centrifuged at 400×g for 5 minutes at 2-8° C., and supernatant aspirated. In the case of freshly isolated cells, the cell suspension in AvO EM or basal medium was centrifuged at 400×g for 5 minutes at 2-8° C. and supernatant aspirated.

### Example 2: Passaging Human Alveolar Organoids Into Single Cell Suspension

To form alveolar organoids, the isolated or cryopreserved cells obtained essentially as described in Example 1 were reconstituted in 50% Matrigel™ (Corning) and seeded as domes in a well of a 24-well cell culture insert of Example 2, such that about 4000 cells were seeded in a 50 μL dome. Such dome cultures could be cultured under expansion and/or differentiation conditions as described below

For expansion, each well comprising a Matrigel dome was exposed as directed by the manufacturer to PneumaCult™ AvO Seeding Medium (SM) (STEMCELL Technologies) for the first 2-3 days, and then cultured in AvO EM (STEMCELL Technologies) for up to 10 days. Culture could be extended to 14 days if more growth is desired. Full medium changes occurred every 3-4 days.

For differentiation, each well comprising a Matrigel dome was exposed as directed by the manufacturer to AvO Differentiation Medium (DM) (STEMCELL Technologies) for up to 10 days. Culture could be extended to 14 days if more growth is desired. Full medium changes occurred every 3-4 days.

To passage organoids in the Matrigel domes, culture medium was aspirated from each well and contacted with 500 μL ACF Enzymatic Dissociation Solution (STEMCELL Technologies). The volume of enzymatic solution was triturated by pipetting up and down approximately 10 times and incubated at 37° C. for 10 minutes. After incubation, each well was again triturated approximately 5-10 times and incubated at 37° C. for 5 minutes. An equal volume of ACF Enzyme Inhibition Solution (STEMCELL Technologies) was added to neutralize the enzyme solution. The full volume was transferred to sterile 15 ml conical or microcentrifuge tubes, and centrifuged at 400×g for 5 minutes at 2-8° C. The supernatant was removed, and the pellet was resuspended in a minimum volume of 250-500 μL of room temperature AvO EM. Live cell counts were performed by conventional means.

### Example 3: Coating Cell Culture Inserts

The following description relates to the preparation of a 24-well sized cell culture insert (i.e. Transwell® culture insert), but with appropriate scale-up or down could readily apply to different formats. A given well of an insert was coated by adding 100 μL of a solution comprising an extracellular matrix protein. In an exemplary embodiment, 100 μL of a 100 μg/mL Collagen III solution was added to a well and incubated at RT for 1.5-3 hours, and both the apical and basolateral sides were washed three times with PBS. Such a well could be used in downstream culture applications, or a second (and so on) extracellular matrix solution could be further added thereto. In an exemplary embodiment where a second extracellular matrix solution was applied, a well of the insert was layered with 100 μL of 10 μg/mL laminin 332 solution, incubated overnight at 4° C., and washed once or twice with PBS or CellAdhere™ buffer before use. Accordingly, a well of an insert may be coated with one or more ECM proteins prior to culturing alveolar or alveolar-derived cells in 2D (e.g. monolayer), and more specifically prior to expanding and/or differentiating and/or maintaining such cells in 2D (e.g. monolayer).

### Example 4: Seeding the ECM Coated Cell Culture Insert

To prepare the ECM coated cell culture insert of Example 3 for receiving the cells of Example 1, and preferably Example 2, a corresponding well of a microplate (that will receive the insert of Example 3) was washed with PBS and 500 μL AvO SM was added thereto (i.e. the basolateral chamber) and 200 μL of the same medium was added into the apical chamber of the insert (i.e. the coated insert of Example 3). Cell concentration, as prepared in Example 1, and preferably Example 2, was adjusted to 30,000 cells in 200 μL medium (i.e. 150,000 cells per ml) before seeding into an apical chamber of the cell culture insert.

Cells seeded essentially as described in this Example 4 were cultured until a more-or-less confluent monolayer formed, either in a submerged condition or in air-liquid interface condition.

For submerged culture, the AvO SM was aspirated after the cells attached and became confluent, and the basolateral chamber of the insert was filled with 500 μL of AvO EM, and the apical chamber of the insert filled with 200 μL of replacement AvO EM. The cultures were maintained for 1-2 weeks with full medium changes in both chambers every 3-4 days.

For ALI culture, the AvO SM was aspirated after the cells attached and became confluent, and the basolateral chamber of the well supporting/underlying the cell culture insert was filled with 500 μL of AvO EM. Cells were cultured for 3-14 cells under such ALI condition with full medium changes every 3-4 days on the basolateral side of the insert. Any leaked medium into the apical chamber was removed to maintain the cells at ALI.

### Example 5: Expanding and Maintaining AT2 Cells

Cells cultured in accordance with Example 4, were assessed for expansion by microscopy, immunostaining, and flow cytometry analysis.

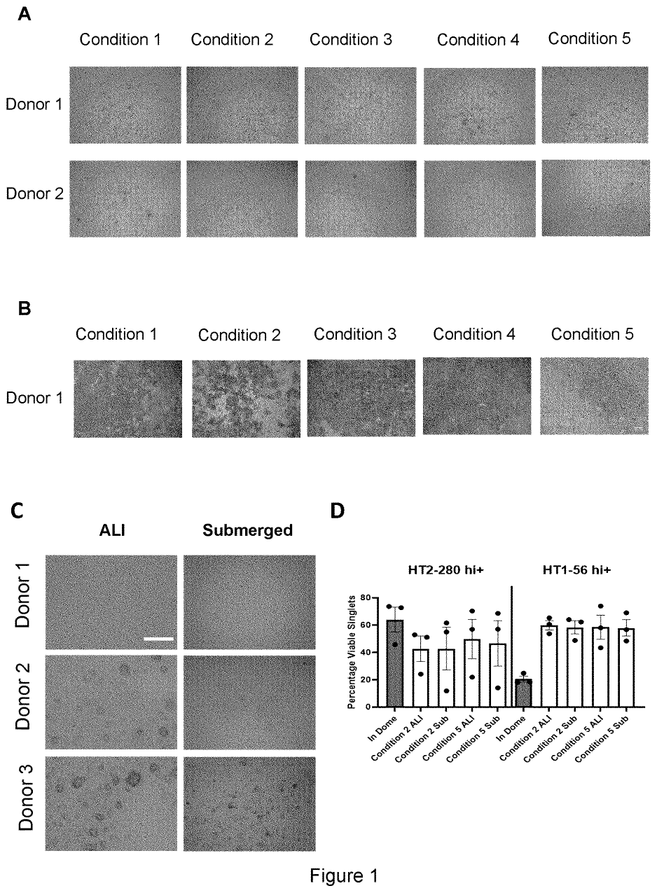

Alveolar epithelial cells from two human donors were expanded in AvO EM in a submerged culture condition for 7 days and microscopic images were taken (FIG. 1A). Various ECM coatings of cell culture inserts were tested, and the ECMs used either alone or in combination were selected from Collagen I, Collagen II, Collagen III, Collagen IV, Vitronectin, Laminin-111, Laminin-521 and Laminin 332. Conditions 1, 2 and 3 represent three different individual ECM proteins, and conditions 4 and 5represent two different pairwise combinations of extracellular matrix proteins. Each of Conditions 1,2, 3, 4, and 5 supported the formation of alveolar epithelial cell monolayers.

In an experiment similar to above, the same ECM conditions (Conditions 1, 2, 3, 4 and 5) were tested in longer term (day 20) expansion/maintenance cultures of submerged alveolar epithelial cells in AvO EM. By day 20 some detachment was observed for all conditions other than Condition 5, which showed preferential maintenance of monolayers (FIG. 1B).

Next, expansion of cells seeded in accordance with Example 4 was compared in submerged and air-liquid interface culture conditions. Alveolar epithelial cells from three different human donors were imaged after 8 days of expansion in AvO EM and in the presence of two ECM proteins (Condition 5). Both culture conditions exhibited comparable formation of confluent alveolar epithelial cell monolayers for all the three donors (FIG. 1C shows representative images of a single donor).

The percentage of viable AT2 marker singlets (HT2-280 hi+) and AT1 marker singlets (HT1-56 hi+) after culturing in the foregoing conditions was assessed by flow cytometry (FIG. 1D). Cells were expanded on either a single ECM (Condition 2) or dual ECM (Condition 5), and in either submerged or air-liquid interface conditions. Expression of indicated markers was compared to a control culture of cells (e.g. alveolar organoids cultured in 3D domes as described in Example 2). While cells expanded comparably in both monolayer conditions (to one another and in comparison to the 3D condition), it appeared that for both the submerged and ALI condition the dual ECM condition yielded slightly better expression of HT2-280 hi+cells compared to the single ECM condition. With regard to HT1-56 hi+cells, all conditions showed a marked increase in cell frequency than the control (“In Dome”) condition.

### Example 6: Differentiating AT2 Cells

Cells cultured in accordance with the differentiation conditions of Example 2 (but otherwise seeded in accordance with Example 4 onto inserts coated essentially as described in Example 3), were assessed for expansion by microscopy, and for marker expression by immunostaining, and flow cytometry.

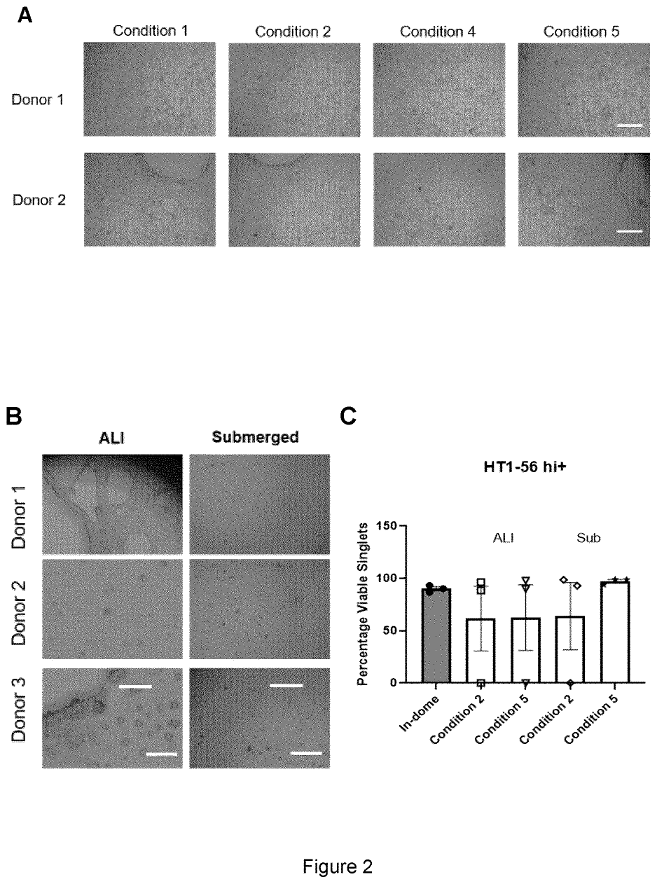

Alveolar epithelial cells from two human donors were seeded onto Conditions 1, 2, 4 and 5 (of Example 5) in a submerged culture condition, and were expanded in AvO EM for 4 days followed by 4 days in AvO DM. Microscopic images (4× magnification) were taken after 8 days (FIG. 2A). Qualitatively comparable differentiation cultures were observed in conditions that included single extracellular matrix protein (Conditions 1 and 2) and in conditions that included two extracellular matrix proteins (Conditions 4 and 5).

Next, differentiation of cells seeded and cultured as described above was compared in submerged and air-liquid interface culture conditions. Alveolar epithelial cells from three different human donors were imaged after the 7-day culture period in the presence of either a single ECM (Condition 2) or dual ECM proteins (Condition 5). The submerged culture condition yielded better cultures than the air-liquid interface condition (FIG. 2B).

The percentage of viable AT1 singlets (HT1-56 hi+) after culturing in the foregoing conditions was assessed by flow cytometry (FIG. 2C). Cells were differentiated on either a single ECM (Condition 2) or dual ECM (Condition 5), and in either the submerged or air-liquid interface conditions, and differentiation was compared to a control culture of cells (e.g. AT1 alveolar organoids cultured in 3D domes as described in Example 2. It was observed that both ECM conditions in either culture condition (e.g. submerged or air-liquid interface) yielded comparable differentiation for two different human donors, and that such differentiated HT1-56 hi+ cells can be maintained for 7 days. In addition, the two ECM condition in submerged culture yielded nearly 100% viable singlets for all three donors tested. Notably, a single donor failed in all conditions other than submerged, condition 5, suggesting that such conditions reduce or limit donor effects (e.g. inter-donor variability).

### Example 7: Immunocytochemistry of Expanded and Differentiated Cell Cultures

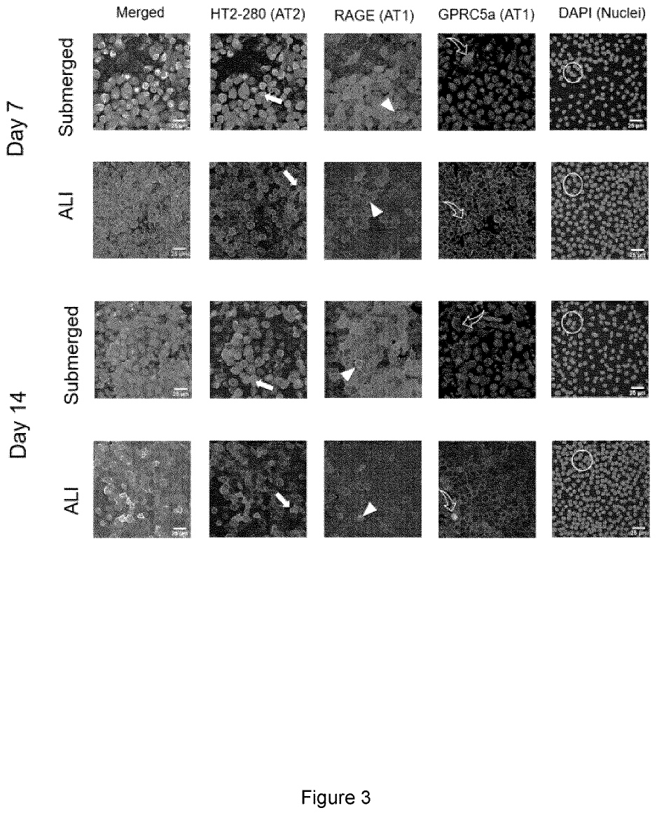

Immunocytochemical staining was performed for AT2 and AT1 cell surface markers on AT2 cells from a single human donor expanded in submerged culture or in ALI for either 7 or 14 days in ECM condition 5 (FIG. 3). Conventional AT2 and AT1 cell markers were used. The results showed expression of AT2 cell marker (HT2-280) and AT1 cell markers (RAGE, GPRC5a) among the cells expanded in submerged culture for 7 or 14 days. The results confirm that AT2 cells can be maintained in submerged culture on two ECM proteins for up to 14 days. It was further observed that AT2 cells from the same donor expanded at ALI for up to 14 days expressed AT2 and AT1 cell markers. The results confirm that AT2 cells can be maintained for at least two weeks in both submerged and ALI culture conditions on two ECM proteins.

1. A method of culturing epithelial cells as an adhered culture, comprising
a) seeding a population of cells onto a surface coated with one or more extracellular matrix proteins;
b) exposing the population of cells of step a) to a cell culture medium; and
c) yielding a culture of epithelial cells in monolayer,
wherein the population of cells are a suspension of cells dissociated from an organoid.

2. The method of claim 1, wherein the surface is a bottom wall of a plate or a membrane of a cell culture insert.

3. The method of claim 1, wherein the one or more extracellular matrix proteins comprise one or more of a collagen, a vitronectin, or a laminin.

4. The method of claim 1, further comprising seeding the population of cells onto a surface coated with two or more individual extracellular matrix proteins.

5. The method of claim 4, wherein the two or more individual extracellular matrix proteins are a laminin and a collagen.

6. The method of claim 3, wherein the laminin is laminin 332, laminin 111 or laminin 521.

7. The method of claim 3, wherein the collagen is collagen type I, type II, type III or type IV.

8. The method of claim 1, further comprising culturing the population of cells submerged in the culture medium or at air-liquid interface.

9. The method of claim 1, wherein the epithelial cells are alveolar epithelial cells.

10. The method of claim 9, wherein the alveolar epithelial cells are alveolar epithelial type 2 (AT2) cells and/or alveolar epithelial type 1 (AT1) cells.

11. The method of claim 1, wherein the population of cells are donor- or PSC-derived.

12. The method of claim 1, wherein the population of cells are cryopreserved or freshly isolated.

13. (canceled)

14. The method of claim 1, wherein the organoid is an alveolar organoid.

15. The method of claim 1, wherein the cell culture medium is an expansion medium.

16. The method of claim 1, further comprising d) differentiating the culture of cells from alveolar epithelial type 2 cells to alveolar epithelial type 1 cells in a differentiation medium.

17. The method of claim 1, further comprising providing a pre-differentiated population of cells before seeding in step a).

18. The method of claim 17, wherein the pre-differentiated population of cells comprise alveolar epithelial type 1 cells differentiated from alveolar epithelial type 2 cells.

19. The method of claim 5, wherein the two or more individual extracellular matrix proteins are coated sequentially.

20. The method of claim 15, wherein the expansion medium comprises one or more cytokines and one or more growth factors.

21. The method of claim 5, wherein the laminin is laminin 332, laminin 111 or laminin 521,and the collagen is collagen type I, type II, type III or type IV.
